(19) United States
(12) Patent Application Publication
(10) Pub. No.: US 2026/0345073 A1
(43) Pub. Date: Nov. 5, 2026

(54) SYSTEM AND METHOD FOR MONITORING CIRCADIAN RHYTHM

(71) Applicant: Resmed Pty Ltd, Bella Vista (AU)

(72) Inventors: Faizan Javed, Sydney (AU); Ross William Deas, Sydney (AU); Kavya Mathur, Sydney (AU); Andrew Chan, Sydney (AU); Japneet Kaur Bindra, Sydney (AU); Jeffrey Peter Armitstead, Sydney (AU); Wouter Jan Pieter Hoogland, Sydney (AU)

(21) Appl. No.: 19/660,641

(22) Filed: Apr. 28, 2026

(30) Foreign Application Priority Data
May 1, 2025 (AU) 2025901612

(51) Int. Cl. A61B 5/00

(52) U.S. Cl. CPC A61B 5/4857; A61B 5/7264

(57) ABSTRACT

A method for monitoring a user’s circadian rhythm, the method comprising acquiring sensor data using one or more monitoring sensors, each configured to measure a secondary physiological parameter or biomarker associated with the user, and inputting the sensor data to a correlation model. The correlation model is configured to correlate the sensor data with a primary physiological parameter or biomarker known to be associated with the user’s circadian rhythm.

### CROSS-REFERENCE TO RELATED APPLICATIONS

The present disclosure is related and claims priority under 35 U.S.C. §119(e), to Australia Provisional Patent Application No. 2025901612, entitled SYSTEM AND METHOD FOR MONITORING CIRCADIAN RHYTHM, to Faizan Javed et al., filed on May 01, 2025, the contents of which are hereby incorporated by reference in their entirety, for all purposes.

### TECHNICAL FIELD

The present disclosure relates generally to systems and methods for monitoring the circadian rhythm of an individual and for establishing a profile of the individual’s circadian rhythm to enable that monitoring.

### BACKGROUND

Sleep plays a crucial role in physical and mental well-being. Despite its importance, many people struggle with sleep-related issues, which can significantly impact their quality of life. Understanding the mechanisms behind sleep can help address these issues and promote better health.

A key regulator of sleep is the person’s circadian rhythm, an internal biological clock that operates on a roughly 24-hour cycle. The circadian rhythm is internally regulated by the suprachiasmatic nucleus (SCN), which controls the release of melatonin, a hormone which influences sleep. It also influences internal biological clocks for other processes in the human body such as sleep stages, including hormone release, body temperature, and digestion.

One of the most important regulators of SCN activity is the lighting environment in which the individual is located, suggesting that for most individuals, the 24-hour sleep-wake pattern is synchronized with the lighting levels within the environment where the person is located. In the population there are differences in the circadian rhythms of different individuals. Aside from the biological differences between different individuals, people may have different lifestyle habits or experience disruptions which may cause a shift or change in the circadian rhythm, such as those caused by shift work, travel across time zones, or exposure to artificial light.

As such, the circadian rhythm of a person is not static but is dynamic and adapts in response to various internal and external factors, thereby adjusting and time-shifting based on changes in the environment and an individual’s lifestyle.

A misalignment between a person’s current circadian rhythm and the required sleep-wake cycle for the person’s circumstances at that time can lead to sleep disorders and other health problems. Therefore, an understanding of a person’s circadian rhythm at particular times is important in gaining understanding of any misalignment and in addressing related issues that may accompany a disruption to circadian rhythm.

### SUMMARY

The present disclosure relates to enabling an improved understanding of a person’s circadian rhythm. Aspects of the current disclosure may include a system for obtaining more accurate information for profiling an individual’s circadian rhythm. This can be helpful in further applications, for example, for developing personalized schedules for therapy to help realign circadian rhythms or to develop adaptive personalized medicine approaches.

In a first aspect, the invention provides a method for monitoring a user’s circadian rhythm. The method comprises:
acquiring sensor data using one or more monitoring sensors, each configured to measure a physiological parameter or biomarker associated with the user; and
inputting the sensor data into a correlation model, the correlation model being configured to correlate the sensor data with a primary physiological parameter or biomarker known to be associated with the user’s circadian rhythm.
In another aspect, the invention provides a system for profiling a user’s circadian rhythm. The system comprises:
   a first sensing arrangement configured to measure a primary physiological parameter or biomarker known to be associated with the circadian rhythm;a second sensing arrangement comprising one or more monitoring sensors, each configured to measure a secondary physiological parameter or biomarker associated with the user;
   a controller configured to process data from the first sensing arrangement and data from the second sensing arrangement, to profile the user’s circadian rhythm by:
      determining one or more correlations between first data acquired using the first sensing arrangement and second data acquired using the second sensing arrangement, the first and second data being acquired over an initialization period;
      monitoring sensor data from the second sensing arrangement over a subsequent period after the initialization period;
      estimating values of the primary physiological parameter or biomarker, based on the determined one or more correlation values and the monitored sensor data from the second sensing arrangement;
      profiling the user’s circadian rhythm, based on a circadian profile established using the estimated values.

The above summary is not intended to represent each implementation or every aspect of the present disclosure. Additional features and benefits of the present disclosure are apparent from the detailed description and figures set forth below.

### BRIEF DESCRIPTION OF THE DRAWINGS

The foregoing and other advantages of the present disclosure will become apparent upon reading the following detailed description and upon reference to the drawings.

FIG. 1 is a schematic representation of a system for establishing a user’s circadian rhythm, in accordance with an embodiment disclosed herein;

FIG. 2 is a schematic representation of an example of the processing performed by the system for establishing a user’s circadian rhythm;

FIG. 3 depicts a method for building a circadian rhythm determination model in accordance with an aspect of the present disclosure;

FIG. 4 is a schematic representation of a user application provided in accordance with an aspect of the present disclosure;

FIG. 5 depicts a data collection process in accordance with an aspect of the present disclosure;

FIG. 6 is a schematic representation of an architecture of an example wearable device which may be used in the present system.

While the present disclosure is susceptible to various modifications and alternative forms, specific implementations and embodiments thereof have been shown by way of example in the drawings and will herein be described in detail. It should be understood, however, that it is not intended to limit the present disclosure to the particular forms disclosed, but on the contrary, the present disclosure is to cover all modifications, equivalents, and alternatives falling within the spirit and scope of the present disclosure as defined by the appended claims.

### DETAILED DESCRIPTION

In the following detailed description, reference is made to accompanying drawings which form a part of the detailed description. The illustrative embodiments described in the detailed description, depicted in the drawings, are not intended to be limiting. Other embodiments may be utilized and other changes may be made without departing from the spirit or scope of the subject matter presented. It will be readily understood that the aspects of the present disclosure, as generally described herein and illustrated in the drawings, can be arranged, substituted, combined, separated and designed in a wide variety of different configurations, all of which are contemplated in this disclosure.

Information in relation to a person’s circadian rhythm may be obtained based on information such as a person’s core body temperature, melatonin levels, sleep stages, heart rate, etc. These parameters are expected to each have a pattern of change which occurs over the circadian cycle, i.e., the cycle in which the circadian rhythm occurs. This pattern may have characteristics such as timing, e.g., peak and trough timings, amplitude, phase, etc. Wearable devices available are marketed to provide one or more of these metrics exhibiting patterns. However, the data from the wearable devices are not necessarily accurate enough to be used to monitor the circadian rhythm. For example, typically, temperature data taken from wearable devices or even thermometers do not necessarily provide enough accuracy or precision to allow the readings to be used as a basis for ascertaining the circadian rhythm of a user of the device. Such data are therefore not necessarily useful for understanding the user’s circadian rhythm, or for assessing the user state, i.e., where the user is within his or her circadian rhythm, at any particular time. Given a person’s internal clock, i.e., circadian rhythm, regulates the production of hormones such as melatonin, it may be possible to measure the levels of the hormones in order to gain an understanding of a person’s circadian rhythm. However, this often involves laboratory testing of the serum levels and may require the individual to attend a clinical location.

The present disclosure relates to improvements to profiling and monitoring a person’s circadian rhythm on an ongoing basis. Aspects of the current disclosure include systems for obtaining more accurate information to assist in this ongoing assessment of a person’s circadian rhythm, i.e., the internal process which regulates various bodily functions. Hereinafter, where used, the term “circadian cycle” will be used to refer to the cycle in which the circadian rhythm occurs. The term “circadian profile” will be used to refer to the pattern(s) exhibited by one or more physiological parameters or biomarker levels, at different times of the day. The circadian rhythm for an individual’s sleep-wake cycle may be inferred from the circadian profile comprising pattern(s) in relation to one or more physiological parameters or biomarker levels.

Described herein are systems for improved profiling of a user’s circadian rhythm based on one or more measurements acquired in relation to the user. The measurements may include physiological or biochemical measurements, or both. Aspects of the disclosure thus also provide methods for collecting the measurement data and profiling and thus monitoring the circadian rhythm from the collected data.

According to some embodiments, the systems and methods described herein implement a two-stage approach comprising an initial calibration phase followed by a proxy inference phase. During the calibration phase, at least one primary physiological parameter or biomarker known to reflect the user’s circadian rhythm (e.g., core body temperature, melatonin level, or cortisol level) is measured using a high‑fidelity sensing arrangement over an initialization period. Concurrently, one or more secondary physiological parameters are measured using one or more monitoring sensors suitable for continuous or long‑term use. A correlation model is generated based on a relationship between the primary parameter data and the secondary parameter data obtained during the calibration phase. During the proxy inference phase, the correlation model is applied to secondary parameter data acquired without continued use of the high‑fidelity sensing arrangement, thereby enabling estimation of the primary parameter and inference of the user’s circadian rhythm over time. This approach allows clinically relevant circadian information to be obtained with reduced reliance on invasive, expensive, or impractical sensing modalities.

Embodiments of the present disclosure establish a personalized mapping between indirectly measured physiological parameters and directly measured biomarkers that reflect a user’s circadian rhythm. Unlike approaches that rely on generalized or population‑based relationships, the correlation model is determined using data acquired from the specific user during an initialization period in which both direct and indirect measurements are obtained. The personalized mapping captures user‑specific physiological characteristics, including variations in amplitude, phase, and sensitivity to behavioral or environmental influences. The mapping is subsequently applied to indirectly measured data acquired after the initialization period to estimate values of the direct biomarker and infer the user’s circadian state over time, thereby improving accuracy and relevance compared to systems that rely solely on indirect measurements without individualized calibration.

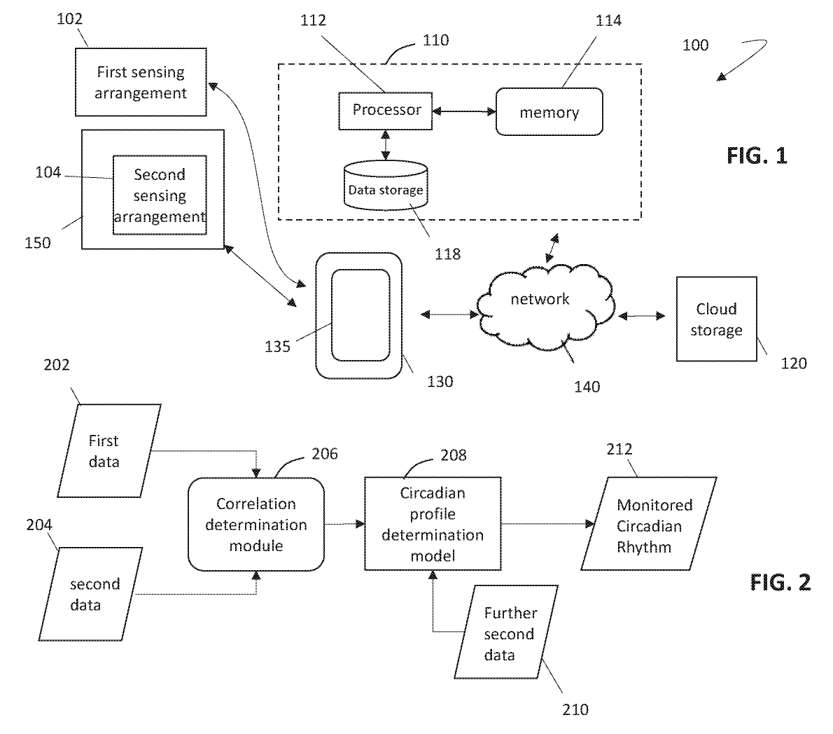

FIG. 1 depicts an example system 100 that may be implemented for collecting measurement data and profiling a person’s circadian rhythm for the collected data. The system 100 in this example comprises a first sensing arrangement 102 and a second sensing arrangement 104. The first sensing arrangement 102 may be configured to acquire reading of a physiological parameter known to be regulated by, and fluctuate with, a person’s circadian rhythm, or of a physiological parameter or known to have levels which fluctuate with an ultradian cycle known to be influenced by the circadian rhythm. Some examples include a person’s core body temperature, or a serum level of one or more hormones known to fluctuate in alignment with a person’s circadian rhythm, such as melatonin or cortisol. The first sensing arrangement 102 is thus configured to measure one or more primary physiological parameters which have a pattern which is driven by a person’s circadian rhythm.

The first sensing arrangement 102 is intended to be used to acquire data for profiling the user’s circadian rhythm during an initialization period. It is configured to be able to accurately measure one or more primary physiological parameters or provide a reliable proxy for the measurement thereof. It may be, for example, an ingestible sensor configured to be swallowed and measure the core body temperature whilst it passes through the user’s digestive system. It may be, as another example, an injectable sensor. The injectable sensor may be biodegradable or bioabsorbable. The first sensing arrangement may comprise a transcutaneous sensor. Depending on the form that the first sensing arrangement 102 takes and the mechanism by which the sensing arrangement is delivered, the first sensing arrangement 102 may comprise wireless transmission capabilities. Non limiting examples of such capabilities include, for example, near field communications (NFC), Bluetooth®, radio frequency (RF), etc.

The second sensing arrangement 104 is configured to measure one or more physiological parameters. The second sensing arrangement 104 is preferably provided by a device or devices which is or are more readily accessible by users, and/or more convenient for acquiring data over a longer period of time compared with the first sensing arrangement 102. Therefore, the second sensing arrangement 104 is intended to provide the data used for the subsequent monitoring and/or further profiling of the user’s circadian rhythm, after the initialization period. The parameters measured may include parameters which exhibit patterns influenced by the circadian rhythm, parameters that exhibit patterns influenced by the circadian rhythm but may also be influenced by other factors, or parameters that do not necessarily exhibit a pattern influenced by the circadian rhythm. Non-limiting examples of parameters which may be measured by the second sensing arrangement 104 may include one or more of heart rate, heart rate variability, oxygen level, ECG, pulse rate, skin temperature, breath temperature, ear temperature, pupil size, polysomnography parameters (such as EEG, ECG, EOG, and EMG and/or parameters derived from such data), blood pressure, perspiration rate, respiration flow rate, and the like. The second sensing arrangement 104 may be provided by a device 150 which includes one or more physiological sensors, configured to acquire the physiological measurements externally. The second sensing arrangement 104 may be provided using two or more devices 150. The device 150 may be a wearable device such as a smart watch, ring, ear-worn device, a patch, mask, etc.

A wearable device 150 may be configured with wireless communication capabilities, such as RF, NFC, Bluetooth®, network connectivity which may use protocols such as 3G/4G/5G, etc. In a particular example, the device 150 providing the second sensing arrangement 104 may be configured to be paired with the first sensing arrangement 102 and receive measurement data therefrom. Two or more wearable devices may be used. For example, a combination of a smart ring, smart watch, and mask may be used, with each being configured and/or positioned to best acquire particular physiological measurements.

The system 100 comprises a controller 110 configured to receive and process the data measured by the first and second sensing arrangements 102, 104. In some embodiments, one of the first and second sensing arrangements 102, 104 includes sensor(s) configured to measure a physiological parameter or the levels of a biomarker which is known to have patterns driven by the circadian rhythm. These are referred to herein as “primary parameters.” Some examples include the core body temperature and the melatonin levels. The other one of the first and second sensing arrangements 102, 104 includes sensor(s) configured to measure physiological parameters and/or biomarkers which are not necessarily known to directly correlate with the body’s circadian rhythm, such as heart rate, skin temperature, etc. These are referred to herein as “secondary parameters.” These secondary parameters may be regulated by a person’s internal body processes, but may also be influenced by external factors, such as stress or environmental factors.

The controller 110 comprises one or more processors 112 configured to perform machine executable instructions stored in a memory location 114 accessible by the one or more processors 112 to process the received data. The controller 110 may also have access to a data storage 118 to store the received information and/or the result from the analysis. The controller 110 is represented in dashed lines, to denote that the aforementioned components may not necessarily be collocated. The data obtained by the first and second sensing arrangements 102, 104 may be stored remotely or in a cloud storage 120. The data storage 118 may store a database used to collect data on a user population to which a user belongs. Such a database may include additional relevant collected data relating to the user that may be accessed by the controller 110 for other analysis such as providing information for activities, avoiding sleep disorders, and nutrition. Preferably any collection or storage of the data will be in compliance with local regulations or privacy standards.

In the implementation depicted in FIG. 1, the data from the first sensing arrangement 102 and second sensing arrangement 104 may be sent to a local computing device 130. The local computing device may be a mobile computing device such as a tablet or smart phone which is in communication with the sensing arrangements 102, 104. The local computing device 130 is configured to transmit the data to the remote controller 110 over a network 140. Analysis results from the controller 110 may be transmitted over the network 140 to be displayed within a user application 135. The user application 135 may be a native application executing on the local computing device 130 or a web application. In another implementation, the data from the first sensing arrangement 102 and second sensing arrangement 104 are transmitted to the local computing device 130 which performs some or all of the processing. In FIG. 1, bidirectional arrows are shown between the local computing device 130 and the first and second sensing arrangements 102, 104. This may be via a local network or a local short or near range connection. The bidirectional communication may not be provided or required in all embodiments, and will be dependent on the implementation and hardware used.

The functions of the local computing device 130 may be performed by a desktop or laptop computer. The local computing device 130 may include one or more processors, a display interface, a user control/input interface (or “I/O interface”), a non-volatile memory/data storage, and a positioning system such as a global positioning system able to determine the user’s location.

In an embodiment according to FIG. 1, the system 100 includes both the local computing device 130 and the wearable device 150. However, it may be possible that in other embodiments, only the wearable device 150 is included, if the wearable device 150 is also configured to perform the tasks required for the specific implementation. The tasks may include, providing the acquired data to the controller 110, executing the user application to provide the required interface to interact with the use, and controlling the sensing arrangement(s) to acquire the sensor data for processing.

It is further contemplated within the scope of the present disclosure that the processing may be performed in a number of different implementations. The specific implementation may depend on factors such as, but not necessarily limited to, connectivity availability and the hardware employed. For example, the processing may be done onboard the wearable device, the local computing device, on another computing device, or one or more servers. Different processing tasks may be distributed across connected devices or servers. Some or all of the processing tasks may be repeated by processors of different devices, e.g., for redundancy. Thus, while the controller 110 is depicted as being separate to the local computing device 130 in FIG. 1, the scope of the present disclosure is intended to cover embodiments where the processing provided by the controller is at least in part performed by the processor(s) of the local computing device 130.

FIG. 2 schematically depicts an embodiment of the processing performed by the system 100. The first data 202 is the data acquired by the first sensing arrangement 102, over a time period. The second data 204 is acquired by the second sensing arrangement 104 over a time period. The second data 204 may be acquired over substantially the same time duration as the first data 202. For example, the second sensing arrangement 104 may be controlled to start data acquisition, or the controller 110 may start to poll the data stream from the second sensing arrangement 104, when the controller 110 starts receiving measurement data from the first sensing arrangement 102. The control may be done automatically by the controller 110 sending a control signal to the first sensing arrangement 102 and/or the second sensing arrangement 104, or it may be triggered by the user, e.g., in an example where the first sensing arrangement 102 is an ingestible sensor, the user may set the second sensing arrangement 104 to start when he or she ingests the first sensing arrangement 102. The controller 110 may choose a starting time and disregard the data samples acquired before the starting time, to ensure the first data 202 and second data 204 being processed are synchronized in time.

The first data 202 is expected to have a pattern which is driven by the circadian rhythm of the user. The controller 110 is configured to execute a correlation determination module 206 to determine a correlation between the second data 204 and the first data 202. The second data 204 may include data acquired by different types of sensors in the second sensing arrangement 104. The determination of the correlation may comprise a determination of which one or ones of the different types of sensor data in the second data 204 best correlate with the first data 202. The determination may comprise a determination of whether any one or ones of the different types of sensor data in the second data 204 do not show any correlation with the first data 202 and therefore should not be considered in monitoring the user’s circadian rhythm. The determination of the correlation may comprise a determination of a combination of two or more of the different types of sensor data in the second data 204 which will correlate with the first data 202. The determination of the correlation may comprise a determination of a whether particular type or types of sensor data in the second data 204, if at a particular range or level, will show a correlation with the first data or will augment the correlation that one or more other types of sensor data will have with the first data. The determination of the correlation may comprise a determination of whether a particular type or types of sensor data in the second data 204, when at particular ranges or levels, should be discounted when monitoring the circadian rhythm.

In some embodiments, the determination of correlation may be made based on statistical methods. In some embodiments, a machine learning model may be used to find the correlation. For example, a neural network model may be trained using the second data 204 as input and the first data 202 as output. The neural network model may be a deep learning model using a neural network. The model may be self-learning, updating its parameters as more data samples of the first data 202 and second data 204 are made available. It may process the first and second data 202, 204 as data streams, during the acquisition of the data or afterwards. The model may preprocess the available samples of the first and/or second data 204 and then use the preprocessing results and/or the raw data to learn the correlation between the first and second data 202, 204. For example, the model may be a neural network model with memory. This is useful for designing a network model that considers not just the physiological parameters being measured but also further information such as variability, change, rate of change, inertia, etc., in the data. An example of this is a model that looks at both the heart rate in the second data 204 and the heart rate variability.

Based on the determined correlation, the controller 110 determines a circadian profile determination model 208 for monitoring the individual’s circadian rhythm based on the input from the second sensing arrangement 104. After the correlation is determined, rather than relying on another “first sensing arrangement” 102, e.g., another ingestible sensor, to acquire the accurate physiological measurements to provide the primary parameter, the second sensing arrangement 104 will be used to monitor the person’s circadian rhythm, using the circadian profile determination model 208. For example, circadian profile determination model 208 may be a parametric model determined by the correlation determination module 206. Or the circadian profile determination model 208 may be the trained machine learning model, trained to estimate a circadian rhythm using the data from the second sensing arrangement as input. Optionally, to limit energy consumption by the second sensing arrangement 104, only the sensor or sensor(s) in the second sensing arrangement 104 determined to have a non-trivial correlation will be used to acquire the physiological measurements. The data which is acquired by the second sensing arrangement 104 after the correlation is determined may be referred to as further second data 210. As the further second data 210 is acquired, it can be processed using the circadian profile determination model 208 to output an estimate for values of the primary parameter that the first sensing arrangement 102 measured. This may be done over time to indirectly monitor the aforementioned primary parameter, i.e., monitoring the estimated values for the primary parameter, using the data from the second sensing arrangement 104 and the circadian profile determination model 208.

By tracking the estimated values over time, the user’s circadian rhythm, as exhibited by the estimated values of the primary parameter, can be profiled and thus monitored (i.e., monitored circadian rhythm 212). For instance, the circadian rhythm may be checked every day using the data from the previous 24 hours. The estimated length of the circadian rhythm may be determined by checking the cycle lengths exhibited in the estimated values.

The pattern exhibited by the values of the primary parameter over time, will be referred to herein as the circadian profile for the primary parameter. It is expected that this circadian profile will be driven by or otherwise linked to the person’s circadian rhythm as can be observed in other processes.

At any one time, by comparing the present primary parameter value estimated based on the sensor data from the second sensing arrangement 104 with the circadian profile for the primary parameter, it may be possible to establish how the current user state compares with an expected user state, i.e., where the user is within the circadian profile associated with the parameter. Deviations between the current user state and the expected user state may signal a disruption or change in the user’s circadian rhythm. The deviations may need to be observed for a minimum period of time for such disruption or change to be inferred or for the data to be further investigated to infer whether a change or disruption in the user’s circadian rhythm has occurred. The deviations may signal hyper- or hypo- activities attributable to stress, physical activities, environmental conditions, dietary or pharmacological intake. The comparison may also enable an assessment of where the user is within his or her circadian rhythm in relation to other processes such as melatonin release, etc., at that time. Therefore, by understanding the circadian profile of the primary parameter, it is possible to gain understanding of user’s circadian rhythm for various processes as well. This can be used by the application to generate recommendations or alerts, such as a notification that there may be disruptions in the user’s circadian rhythm, a recommendation of when to start avoiding blue light exposure, and a recommendation of an optimal bed time as can be deduced from when the user’s melatonin level is expected to peak. An understanding of the user’s circadian rhythm for one or more processes may also be used to manage expected schedule changes, such as those caused by jet lag, travel, or shift work.

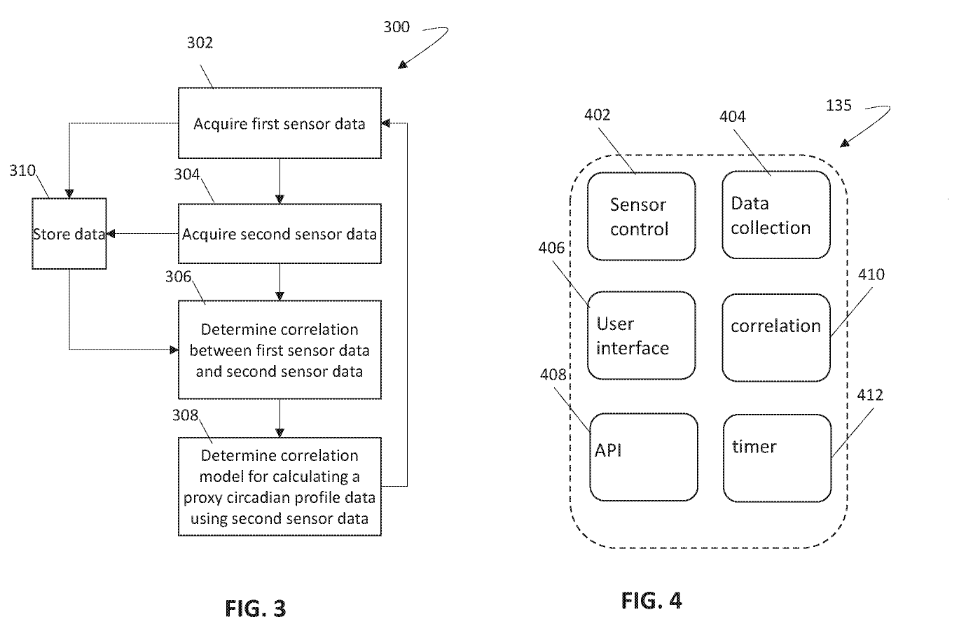

FIG. 3 depicts an embodiment of a method 300 for building a circadian profile determination model 208 configured to ascertain a person’s circadian profile for the primary parameter, estimated based on data from one or more sensors from the second sensing arrangement 104 acquiring physiological data. At step 302, the first sensor data considered to provide an accurate proxy or measurements of a primary parameter acquired. At step 304 the second sensor data is acquired using a second sensing arrangement. The two steps 302, 304 need not occur in the order depicted, in the sense that the initiations of the data acquisition for the first and second sensor data need not be in the depicted order. They may be initialized in any order or at the same time. What is required is that the first and second sensor data both include data acquired over at least a common time period, so that the first and second sensor data acquired over this time period can be processed to determine the correlation. At step 306, the data acquired over this common time period is used to determine a correlation between the second sensor data and the first sensor data. At step 308, the determined correlation may be expressed as a correlation model for estimating the individual’s circadian profile associated with the primary parameter, using his or her personalized physiological measurements acquired using the second sensing arrangement. Thus, the circadian profile being monitored in this process is the circadian profile associated with the primary parameter, using the direct measurements (first sensor data) or using estimated values calculated from the second sensor data once the correlation has been determined. As the circadian profile is related to the user’s circadian rhythm, by monitoring the circadian profile, information regarding the user’s circadian rhythm can be obtained.

The acquired first and second sensor data are preferably stored at step 310. At a later time, newly acquired first and second sensor data may be used to validate or update the correlation model. In some implementations, both the newly acquired data and the historical (stored) data may be used to redetermine the correlation model, and this can serve to tune the model and update it where needed to ensure the model is correct. This can serve to validate a previously determined correlation model, or as a way of further tuning the model for the individual. The validation or tuning may be scheduled to occur regularly. Alternatively or additionally, it may be triggered to occur by specific events, such as travel, the user’s schedule change, illness, relocation, reported behavioral changes, etc.

Referring back to FIG. 1, the stored data may be saved into a user population database, which may be located in the data storage 118, e.g., at a server, or may be stored in cloud storage 120. This allows a server to perform further population-based analyses. The populations may be divided into sub-groups based on information such as user location, gender, age, behavioral categories (e.g., self-described earlier risers), etc. This may be useful for establishing a population or sub-population-based baseline circadian profiles. It may be useful for the purpose of establishing a population or sub-population-based correlation between the expected circadian profile and the indirect measurements provided by the physiological sensors in the second sensing arrangement 104. In the absence of historical data from the user, the baseline circadian profile may be a preset profile. The preset profile may be established using information such as the user’s location, and one or more other parameters such as the user’s age, self-reported chronotype and other self-reported information such as average wake-up times and bedtimes, may be used. Baseline population- or sub-population-based profiles may be used to prepare an initial correlation model, which can be further personalized when the first and/or second data of the individual user becomes available.

The first sensing arrangement 102 may be used during an initialization period, along with the second sensing arrangement 104. The first sensing arrangement 102 tends to be less practical for longer term monitoring of a person’s circadian rhythm, but is considered to acquire data that more accurately reflect a person’s circadian rhythm. On the other hand, the second sensing arrangement 104 may be something which is comparatively more practical for long-term monitoring. For instance, the second sensing arrangement 104 may be provided by sensors in a consumer grade device, such as a wearable device, which can be worn by the user for longer periods of time. The correlation determined from the data will determine how the data measured by sensors provided in the second sensing arrangement correlates with the user’s circadian profile (reflected by the values of the measurement data from the “first sensing arrangement” over time). After the initialization period during which the correlation is determined, the data from the wearable device can then take over, i.e., the circadian profile of the primary parameter previously monitored using the first sensing arrangement 102 will now be monitored using the wearable device without using the first sensing arrangement. For example, the correlation model may be used to, by using the second sensor data as an input, determine a proxy or an estimation for the user’s circadian profile for the primary parameter, thus gaining an understanding of the user’s circadian rhythm.

The correlation determined during the initialization may be validated and/or updated at a later time. If the previously determined correlation is still valid, then it is expected that over the later time period, the second sensor data acquired will still have the same or substantially the same correlation with the first sensor data acquired over that later time period. For example, validation may be performed by acquiring the first sensor data and the second sensor data again, applying the previously determined correlation model to the second sensor data, and seeing whether this yields the expected result, i.e., an output that tracks the acquired first sensor data. If it is found that the correlation model does not correctly produce an output from the second sensor data that aligns with the first sensor data, then the correlation model will be updated by re-running the correlation determination module. The correlation determination module may be re-run using all available first and second sensor data, including historical data from data storage and the new data acquired during a validation period.

In some embodiments, it will be understood that the correlation model needs not necessarily be such that the output from applying the correlation model to the second data will match the circadian profile for the primary parameter. In profiling the circadian rhythm of a person, the timings of various features such as the peak, fall, rise, and trough are of importance, as this information can still provide insight as to the person’s circadian rhythm. Therefore, in some embodiments, the goal of applying the determined correlation to the second data is so that it produces an output that tracks or aligns with the first data, e.g., this may mean that the output from the correlation model will have peaks and troughs that occur at the same or about the same times as those in the first data. Preferably, the waveform obtained by plotting the output of applying the correlation model to the second data over time will generally track the shape of the waveform obtained by plotting the first data over time. Given the first data is considered driven by a circadian rhythm of the user, this provides a way of using the second sensing arrangement to profile the circadian rhythm.

The collection of the first sensor data and/or the second sensor data, particularly in embodiments designed to allow the users to self-test at home, as described above, may involve the use of personal mobile devices such as smartphones and/or wearable technology such as a smart watch via the user application 135.

The user application 135 may be provided as an executable program running on a local computing device 130 (or an alternative device such as a laptop), and/or the wearable device 150. A user application for the local computing device 130 and a compatible application for the wearable device 150 may be provided. The arrangements of the functionalities enabled in the devices, via the respective applications, may be designed based on factors such as hardware capabilities, screen sizes which define the sizes of the display interface, etc., and may be variable in different implementations. For conciseness, the term “user application” 135 is intended to collectively refer to the application program for the user to operate the system 100. It may be provided via an application running on just the local computing device 130, an application on the wearable device, or both.

The user application 135 may be configured to coach the user through a data collection process to obtain the data required to determine the correlation model. The user application 135 may provide a user interface configured to cause a display of instructions for operating the sensing arrangements 102, 104. Some of the display for the user application may be generated and displayed on the wearable device 150. The user application 135 may be configured to provide a data collection module which, when initiated, runs through a program which outputs alerts to notify the user to take the required step(s).

FIG. 4 depicts an example of the user application 135, which may comprise component applications for performing different functions. The component applications which cooperate with each other may be located on different devices (e.g., the mobile device and the wearable device). Instances of the user application or components thereof may be running on both the mobile device and the wearable device. The different instances may be differently configured for the respective devices based on processing and/or hardware specifications. It may comprise a sensor control module 402 for providing control signals to one or more sensor(s) from the first and/or second sensing arrangements 102, 104. This may be within, or cooperating with, a data collection module 404 configured to control the data collection process. The data collection module 404 may cooperate with a user interface module 406 to output instructions or notifications as needed in relation to the data collection process. The user interface module 406 may also be configured to receive input from the user. The user application 135 may comprise an application program interface (API) 408 to communicate with another processor or a server such as a backend server (not shown). For instance it may be configured to receive processing results, updates, etc., from a backend server. It may be configured to send sensor data to the backend server for processing, or send local processing results to the backend server. The user application 135 may include a correlation determination module 410 in embodiments where the correlation is determined locally. A timer module 412 may be provided to associate the time of day with the data. The timer module may be configured to keep track of time as needed during the data collection, or to keep track of preset time periods as required.

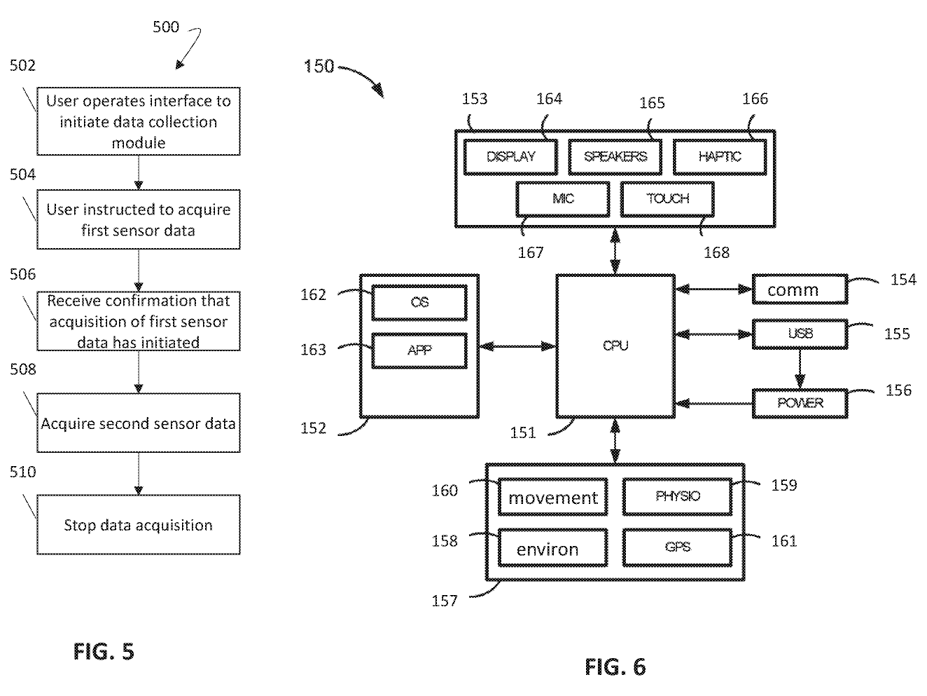

FIG. 5 depicts an example of a data collection process 500, where control of the first and/or second sensing arrangements are triggered or provided using the data collection module 404. In this example, the user (or a carer or clinical staff) will operate an I/O interface provided on the computing device on which the user application 135 is executing, to complete the required data collection. At step 502, the user operates the I/O interface to start the data collection module. At step 504, the data collection module instructs the user to operate the first sensing arrangement to start the collection of the first sensor data. The data collection module may display an interface for the user to confirm the first sensing arrangement 102 being used, before displaying the instruction. At step 506, the data collection module receives a confirmation of the initiation of the operation of the first sensing arrangement 102. This confirmation may be provided by the user, e.g., the user operates the I/O interface to confirm to have swallowed an ingestible sensor. The confirmation may be the receipt of first sensor data from the first sensing arrangement. The confirmation may trigger the data collection module to trigger the local computing device 130 to pair with the first sensing module to detect incoming data from the first sensing arrangement. Alternatively, the pairing may be initiated when the data collection module is launched. At step 508, the second sensor data is acquired. This may be an automatic process, for example, where the confirmation at step 506 automatically triggers the data collection module to control the second sensing arrangement 104 to start sensing. This may be a manual process, for example, where the user is instructed to operate a user input/output interface such as a button, a touchscreen display, etc., to start the second sensing arrangement 104. After a preset amount of time has elapsed or when the first or second sensor data is no longer detected, at step 510, the data collection is stopped. This may involve the data collection module controlling the first, second, or both sensing arrangements to stop, depending on the sensing arrangements used and the implementation. If the stop of the data collection is triggered by the user or by a data loss, the data collection module may be configured to determine whether enough time has elapsed such that sufficient data has been collected in order for the correlation to be determined. If not, it may output a notification for the user to reinitiate the process 500.

It will be understood there may be variations in the steps. For instance, in some embodiments, the collection of one of the first or second sensor data requires user action, e.g., swallowing an ingestible pill, applying a wearable device, etc. The start of the data collection process may involve the data collection module 404 initiating control to cause one of the first or second sensing arrangement 102, 104 to start acquiring the sensor data, and then output an instruction to the user to take the required action to start the data collection using the other one of the first or second sensing arrangement. In another example, the second sensing arrangement 104 may be configured to continuously monitor the physiological parameters. In this case, step 508 will not be separately required.

In one embodiment, the first sensing arrangement 102 is configured to measure core body temperature of the user. The first sensing arrangement 102 may be in the form of an ingestible sensing arrangement, such as an ingestible pill. The sensing arrangement 102 is expected to continue sensing the core body temperature data as it travels through the user’s digestive track, wirelessly transmitting the core body temperature measurement data in the process. Being internal within the user’s digestive tract, the first sensing arrangement 102 is expected to be able to accurately measure minute changes in the user’s core body temperature values, and therefore provide data that will be useful to establish the circadian profile of the core body temperature values.

The transmission may be via, e.g., Bluetooth®. The user may be instructed to first pair the ingestible sensing arrangement 102 with his or her mobile device (i.e. local computing device) 130 before ingesting the pill. In some embodiments, the first sensing arrangement 102 may comprise multiple such pills. The user may be instructed to take the pills at regular time intervals (e.g. one every 4 hours or 6 hours), to ensure enough data may be obtained. The user application 135 may be configured to cause a display to alert the user to take another pill if it ceases detecting data from a paired, ingested pill. The user application 135 may be configured to cause an alert to be output, to remind the user to take the pill if the application does not detect any data, and to help encourage user compliance. For example, the alert generation process may be tied to both a timer process and the data process. If a preset amount of time has elapsed since a starting time, but no data has been received, then the alert may be generated. The starting time may be an installation time of the user application, a time that the user is expected to have ingested a pill, or the most recent time that data was recorded. The number of ingestible pills and the timing for ingesting the pills should preferably be set to ensure user comfort and safety.

The first sensing arrangement 102 which is used during the initialization period, or one or more subsequent periods where the first data is re-acquired for the purpose of updating or validating the correlation model, may not necessarily need to be an ingestible, injectable, or transcutaneous sensing arrangement. Other forms of sensing arrangements or methods may be used. A combination of different forms or methods of acquiring the first sensor data may be used during any measurement period. Different arrangements may be used for acquiring the “first sensor data” during different measurement periods, e.g., during the initialization and any subsequent periods.

Furthermore, rather than tracking biomarkers that are driven by the circadian rhythm, the first sensor data may be obtained by monitoring the levels of biomarkers from body processes that have ultradian cycles which are affected by the circadian rhythm of the user. Alternatively, data in relation to biomarkers or other parameters that are driven by the circadian rhythm, as well as one or more of those which are considered to have ultradian cycles influenced by the user’s circadian rhythm, may both be used. Thus, it can be envisaged that some types of sensors may be useful for monitoring parameters related to both the user’s circadian rhythm and the ultradian cycles which are influenced by the circadian rhythm. If different types of data are included in the first sensor data, it is possible for different types of sensors to be employed in the first sensing arrangement to acquire the different types of data.

Within the general concepts mentioned above, in one example, the first sensing arrangement 102 may be configured to measure saliva melatonin levels which, given enough sensor sensitivity and a useful signal to noise ratio, can provide a clinically useful proxy for serum melatonin levels. For example, the first sensing arrangement may be provided in the form of a kit, to be used for an individual to detect the saliva melatonin levels, in order to obtain information on his or her circadian rhythm. This allows the collection and monitoring to be done either at home or in a clinical setting. Such a kit may include a saliva sample collector, such as a cup, straw, chewing gum, swab, an oral-device such as a mouth guard, or tooth brush. The kit may include a reader configured to measure the saliva melatonin levels in the sample, and to transmit the data to a user computing device, such as the aforementioned local computing device 130.

The reader may comprise a detection device for detecting melatonin and/or measuring melatonin levels. The detection device may be any device suitable for measuring melatonin in a sample. In one embodiment, the detection device is an immunoassay device comprising at least one anti-melatonin antibody, typically an anti-melatonin monoclonal antibody (mAb), or a melatonin-binding fragment thereof. Lateral-flow immunoassay cassettes can be readily used to obtain a qualitative result relating to the presence and level of melatonin. Lateral flow immunoassay cassettes can be used to obtain quantitative or qualitative assessment of melatonin levels. In one embodiment, the lateral flow immunoassay cassette comprises a diffusible strip which comprises a sample zone for receiving a saliva sample, a capture zone comprising an anti-melatonin antibody or melatonin-binding fragment thereof, typically fixed to the diffusible strip, and a diffusible labeled reporter antibody capable of binding the melatonin bound to the anti-melatonin antibody or melatonin-binding fragment thereof. Suitable labels include visible and fluorescent dyes, latex beads, enzymes, gold nanoparticles, silver nanoparticles, titanium nanoparticles, europium fluorophores, quantum dots, and the like. In one embodiment, the immunoassay device is a lateral flow immunoassay cassette. A saliva sample is applied to the sample zone, and liquid in the sample carries the melatonin through the diffusion strip into the capture zone comprising the anti-melatonin antibody or melatonin-binding fragment thereof, where the anti-melatonin antibody or melatonin-binding fragment thereof binds the melatonin in the sample when present. Calibration standards can be included in the cassette to provide a detectable signal that correlates to known concentrations of melatonin. For example, one of more calibration standards may include a known amount of melatonin that is pre-bound to the diffusion strip. Binding of the melatonin to the anti-melatonin antibody or melatonin-binding fragment thereof is detected when the labeled reporter antibody binds to the melatonin and the calibration standards in an amount proportional to the amount of melatonin present. Concentration of the reporter antibody where the melatonin has been captured results in a detectable signal, which can be used to determine the concentration of melatonin in the sample. In another embodiment, the lateral flow immunoassay cassette may comprise a competitive immunoassay. In one example of a competitive immunoassay, labeled melatonin is bound to the anti-melatonin antibody or melatonin-binding fragment thereof in the capture zone, and the level of melatonin in the sample can be determined though displacement of the labeled melatonin from the anti-melatonin antibody or melatonin-binding fragment thereof by unlabeled melatonin in the sample. The labeled melatonin may be labeled with any label which is suitable for competitive immunoassay with the melatonin. Alternatively, the collector may itself be configured to include sensors or readers to measure the melatonin level in the sample.

In embodiments where the data collection is done at separate times, the user (or a carer or clinical staff) may be instructed, e.g., via the user application 135 (or a corresponding carer or clinical application), to perform the sample collection at certain times during the day, in order to obtain samples over a preset period. For example, the user (or a carer or clinical staff) may be instructed to collect the sample at set intervals during a period of time which covers the day-time hours in the time zone at the user’s location, which may be determined using, e.g., a GPS or may be read from the date and time data from the local computing device. Preferably, the collection period will be long enough to cover a peak and a trough of the user’s 24 hour (or roughly 24-hour) circadian cycle for the measured parameter. In clinical settings, the collection period may be longer, e.g., 24 hours or longer, as some users may have circadian cycles which are longer than 24 hours. The collection times may be closer together over certain periods during the measurement period, e.g., during a period of time when the trough or peak of the circadian cycle is expected to occur.

If the data collector is something which is worn or applied continuously over the period, e.g., a mouth guard, the measurement may, though is not required to, be done over a period covering at least the night time hours, to minimize the inconvenience to the user.

The data collection system may be configured to initiate data collection using the wearable sensors (e.g., polling the data stream from the wearable sensors) prior to taking the samples, to ensure there will be measurement data covering the same time period over which the samples are taken. The user may be instructed to confirm sample collection is completed in the user application 135 in order to stop the data collection by the wearable sensors.

In another example, the first sensing arrangement 102, providing the direct measurements of biomarkers or physiological parameters aligned with circadian or ultradian cycles, is configured to measure cortisol levels. The cortisol may be detectable in sweat. During an initialization period, the user may be provided with a cortisol monitoring apparatus. This may be, e.g., a pad sensor which is worn on the user’s body and collects sweat, and detects the cortisol level in the collected sweat.

An example of a pad sensor for measuring cortisol levels is described in, for example, Yeasman et al. 2023, Enzyme-mimics for sensitive and selective steroid metabolite detection. ACS Appl. Mater. Interfaces, Vol 15(11); 13971-13982. Briefly, this document describes an integrated electrochemical biosensor comprising a catalytic film over one or more working electrodes. The catalytic film comprises electrocatalyst molecules embedded in a molecularly imprinted polymer (MIP) matrix which comprises shape-selective cavities for high-affinity selective binding of cortisol. The electrocatalyst molecules are located in close proximity to where cortisol molecules are trapped within the shape-selective cavities within the MIP matrix. When cortisol is trapped in the shape selective cavities, the electrocatalyst molecules reduce ketone groups of the cortisol to generate an electrochemical response.

The cortisol monitoring apparatus may be configured to transmit the cortisol level measurements to the user application 135. The pad sensor may be provided in a wearable device. For example, it may be included in the wrist band for a smart watch, a standalone wrist band, a ring, an arm band, a belt or waist band, an eye mask, etc.

Given the cortisol levels may be affected by the user’s emotional state, stress level, state of agitation, etc., the user may be instructed to wear the sensor over a long enough measurement period, e.g., multiple days, so that there is enough data to provide a longitudinal baseline which is observable in spite of potential temporary variations in the cortisol levels read during the measurement period. The user may be instructed to wear the sensor at least during nighttime hours at the user’s location, to monitor the sweat cortisol levels during sleep.

Optionally, the first sensing arrangement 102 may be further configured to measure a core body temperature over time, e.g., using an ingestible sensor, during a period which at least partially overlaps with the measurement period over which the cortisol levels are read. A correlation between the core body temperature reading and the cortisol levels can be determined, e.g., using the methodology described above in relation to FIG. 2. This may help to further eliminate or reduce the effect of temporary variations in the cortisol level.

Optionally, one or more of physiological parameters, movement parameters, and environmental parameters are also measured whilst the sweat cortisol levels are being measured. Some examples include heart rate variability data, EEG, and changes in the body movement (e.g., as measured by one or more accelerometers or EMG). The sensors for measuring these parameters may be provided by a wearable device. This may be a wearable device providing the second sensing arrangement. These can be processed to determine further information which may be relevant in identifying whether there may be temporary spikes in the measured cortisol levels. The further information may be in relation to behavioral or emotional markers, such as agitation, exercise, or in relation to environmental information (e.g., temperature, humidity). This may be used by the system to assess whether the variations observed in the measured cortisol levels are attributable to the circadian fluctuations or are due to other factors such as the user experiencing stress or the user’s physical activity.

Once the circadian profile of the user’s sweat cortisol level is established, the circadian rhythm as exhibited in this profile is also established. The cortisol level measurements that exhibit the circadian rhythm may be considered to provide the baseline cortisol measurements for the user. Therefore, the circadian profile may further provide useful information in relation to the level of sweat cortisol that is expected at any time. For example, further cortisol level measurements may be acquired and compared with the baseline cortisol level to monitor the user’s state of stress. For example, if the sweat cortisol level measured at time T is higher than the expected baseline cortisol level at that time, this may indicate that the user is experiencing stress. This information may be output as a notification or alert on the user application 135.

In the above, it is mentioned that the second sensing arrangement is typically configured to include sensors which measure “indirect” biomarkers. However, it is possible for both the first and second sensing arrangements to be the type which provide measurements of “direct” biomarkers. For example, the aforementioned saliva melatonin or sweat cortisol detection apparatus could be used as a second sensing arrangement. It may be further possible for the second sensing arrangement 104 to include sensing apparatuses to sense both “direct” and “indirect” parameters. For example, the second sensing arrangement 104 may include both a saliva melatonin detector and a wearable sensor. In some examples, if the readings from the saliva melatonin detector do not have a sufficient correlation with the first sensor data, then the determination may be done again, but this time using data from the wearable sensor instead of or in addition to the saliva melatonin reading.

The physiological data to combine with the “direct” parameter data may be chosen depending on the primary parameter which is measured. For example, when the biomarker being measured is cortisol, the cortisol level reading may be combined with the readings of physiological parameters known to be aligned with cortisol level fluctuations, such as heart rate, heart rate variability, blood pressure, core body temperature, pupil size, etc. The combined data may be used to determine a proxy for the user’s circadian rhythm. The selection of the physiological parameter(s) to be used for this combination may depend on how convenient and/or invasive it is to acquire the data.

In embodiments of the system described herein, additional measurements which are not associated with the user’s biomarkers or other physiological parameters may be acquired, such as movement data (e.g., data from one or more inertia sensors, GPS data), user-entered exercise data, and environmental data (e.g., humidity, temperature), and may also be considered. These additional measurements may help to identify temporary elevations or depressions in particular physiological measurements which are separate from the fluctuations which may be caused by the intrinsic circadian rhythm in the person’s sweat cortisol level. The algorithm for determining the correlation between the first sensor data and the second sensor data may be configured to account for the influence of any identified temporary elevations or depressions, for an improved correlation determination. This is alluded to above in relation to the example involving the monitoring of the sweat cortisol level.

In the embodiments mentioned above, the system comprises the first and second sensing arrangements. However, in other embodiments, the system 100 may include only the first sensing arrangement, or build a circadian profile relating to a primary physiological parameter associated with the user, relying on data from only the first sensing arrangement. For example, given accurate enough core body temperature readings, or accurate enough saliva melatonin levels, acquired over a long enough period of time such that the trough and peak of a circadian profile may be observed, the observed circadian profile may provide a useful indication of a person’s circadian rhythm. The measured circadian profile may be stored and re-checked at a later time.

An example architecture for the wearable device 150 mentioned in the above is represented in the block diagram shown in FIG. 6. The wearable device 150 includes a processor (such as central processing unit (CPU) 151), a memory 152, a user input/output interface 153, a communication interface 154, a connector interface 155, a power source 156, and a plurality of sensors 157. These may include environmental sensors 158, physiological sensors 159, movement sensors 160, and a global positioning system (GPS) receiver 161.

The memory 152 may be flash memory or other semiconductor memory (e.g., DRAM, SRAM). The memory 152 stores an operating system 162 executed by the processor 151 for operating the wearable device. The memory 152 may store one or more wearable applications that may be executed by the processor 151 including a wearable application 163. The wearable application 163 may be configured to communicate and cooperate with the user application 135 in the local computing device 130.

The user input/output interface (or “I/O interface”) 153 allows the user to provide inputs and receive outputs from the wearable device 150. The user I/O interface 153 may comprise one or more of a display 164, speakers 165, and a haptic actuator 166. The display 164 may be a compact display such as an LCD (liquid crystal display), LED (light-emitting diode) display, or an OLED (organic light-emitting diode).

The user I/O interface 153 may include one or more input devices such as a microphone 167 and a touch sensor 168. The touch sensor 168 may be a capacitive sensor array with the ability to localize contacts to a particular point or region on the surface of the sensor. The touch sensor 168 may be overlaid over the display 164 to provide a touchscreen interface.

The processor 151 controls the operation of the wearable device 150 and executes the wearable programs stored in the memory 152. The processor 151 executes the operating system (OS) 162 and various applications for interfacing with a host device such as the mobile device 130 in FIG. 1. The processor 151 may be configured to execute machine readable instructions to provide the user application 135 provided by the present system 100 to acquire measurement data and estimate a user’s circadian rhythm.

The communication interface 154 is configured to allow wearable device 150 to communicate wirelessly using different protocols with various host devices. The communication interface 154 may include RF transceiver components such as an antenna and supporting circuitry to enable data communication over a wireless medium, e.g., using Wi-Fi (IEEE 802.11 family standards), Bluetooth® (a family of standards promulgated by Bluetooth SIG, Inc.), or other protocols for wireless data communication. The communication interface 154 may provide near-field communication (“NFC”) capability to support wireless data exchange between devices over a very short range. The connector interface 155 is configured to allow the wearable device 150 to communicate with various host devices via a wired communication path. In this example, the connector interface 155 is a Universal Serial Bus (USB) port. The connector interface 155 may also provide a power port, allowing the wearable device 150 to receive power. The connector interface 155 may also provide connections for audio and/or video signals, which may be transmitted to or from a host device in analog and/or digital formats.

The power source 156 provides power for the wearable device 150. In this example, the power source 156 is a rechargeable battery and circuitry operable to charge the battery when the connector interface 155 is connected to a power source.

The sensors 157 provide digital signals to the processor 151. The signals may be provided on a streaming basis or in response to polling. In the depicted example, the sensors 157 include physiological sensors 159 configured to measure physiological parameters, one or more movement sensors 160 such as an accelerometer and a gyroscope. The sensors 157 may also include a GPS receiver 161. The sensors 157 may further include one or more environmental sensors such as a temperature sensor or a humidity sensor. The movement sensors 160 and GPS receiver 161 provide information about the location and/or motion of wearable device 150. Other sensors can also be included in addition to or instead of these examples. For example, a sound sensor can incorporate the microphone 167. The physiological sensor 159 may include sensors such as heart rate sensors, oxygen level sensors, ECG sensors, pulse rate sensors, or the like.

The present disclosure is described with reference to the attached figures, where like reference numerals are used throughout the figures to designate similar or equivalent elements. The figures are not drawn to scale and are provided merely to illustrate the instant disclosure. Several aspects of the disclosure are described above with reference to example applications for illustration. The present disclosure is an example or illustration of the principles of the present disclosure, and is not intended to limit the broad aspects of the disclosure to the embodiments illustrated.

One or more elements or aspects or steps, or any portion(s) thereof, from one or more of any of the claims below can be combined with one or more elements or aspects or steps, or any portion(s) thereof, from one or more of any of the other claims or combinations thereof, to form one or more additional implementations and/or claims of the present disclosure.

While the present disclosure has been described with reference to one or more particular embodiments or implementations, those skilled in the art will recognize that many changes may be made thereto without departing from the spirit and scope of the present disclosure. Each of these implementations and obvious variations thereof is contemplated as falling within the spirit and scope of the present disclosure. It is also contemplated that additional implementations according to aspects of the present disclosure may combine any number of features from any of the implementations described herein.

In the claims which follow and in the preceding description of the technology, except where the context requires otherwise due to express language or necessary implication, the word “comprise” or variations such as “comprises” or “comprising” is used in an inclusive sense, i.e., to specify the presence of the stated features but not to preclude the presence or addition of further features in various embodiments of the technology.

1. A method for monitoring a user’s circadian rhythm, the method comprising:
acquiring sensor data using one or more monitoring sensors, each configured to measure a secondary physiological parameter or biomarker associated with a user; and
inputting the sensor data into a correlation model, the correlation model being configured to correlate the sensor data with a primary physiological parameter or biomarker known to be associated with the user’s circadian rhythm.

2. The method of claim 1, comprising using the correlated sensor data to monitor a circadian profile relating to the user’s circadian rhythm.

3. The method of claim 1, comprising using the correlated sensor data to identify a user state within a user’s circadian profile.

4. The method of claim 2, wherein the circadian profile is determined from measured or estimated values of the primary physiological parameter or biomarker.

5. The method of claim 1, further comprising establishing the correlation model during an initialization period, wherein establishing the correlation model comprises:
acquiring primary parameter data over at least the initialization period, the primary parameter data being measurement data for the primary physiological parameter or biomarker;
acquiring monitoring sensor data using the one or more monitoring sensors over at least the initialization period;
determining one or more correlations between the primary parameter data, and the monitoring sensor data acquired over the initialization period; and
determining the correlation model based on the one or more correlations.

6. The method of claim 1, further comprising profiling the user’s circadian rhythm by:
acquiring data in relation to a primary parameter known to be associated with the user’s circadian rhythm, using an initializing sensor configured to measure the primary parameter, over an initialization period;
acquiring monitoring sensor data in relation to one or more secondary parameters using one or more monitoring sensors, over the initialization period;
determining one or more correlations between primary parameter data, and the monitoring sensor data acquired over the initialization period, to calculate a correlation model; and
profiling the user’s circadian rhythm using monitoring sensor data acquired after the initialization period and the one or more correlations.

7. The method of claim 1, comprising storing data acquired during an initialization period, and updating the correlation model by:
acquiring primary parameter data over at least a model review period;
acquiring monitoring sensor data using the one or more monitoring sensors, over at least the model review period;
determining one or more correlations between the primary parameter data from the initialization period and from the model review period, and the monitoring sensor data from the initialization period and from the model review period; and
determining an updated correlation model based on the one or more correlations determined using the stored data and the primary parameter data and monitoring sensor data acquired over the model review period.

8. The method of claim 7, wherein the primary parameter data comprises at least one of core body temperature data of the user, measurements of saliva melatonin levels of the user, or measurements of sweat melatonin levels of the user.

9. A system for profiling a user’s circadian rhythm, comprising
a first sensing arrangement configured to measure a primary physiological parameter or a biomarker known to be associated with a circadian rhythm;
a second sensing arrangement comprising one or more monitoring sensors, each configured to measure a secondary physiological parameter or biomarker associated with a user;
a controller configured to process data from the first sensing arrangement and data from the second sensing arrangement, to profile the user’s circadian rhythm by:
determining one or more correlations between first data acquired using the first sensing arrangement and second data acquired using the second sensing arrangement, the first and second data being acquired over an initialization period;
monitoring sensor data from the second sensing arrangement over a subsequent period after the initialization period;
estimating values of the primary physiological parameter or biomarker, based on the determined one or more correlation and the monitored sensor data from the second sensing arrangement; and
profiling the user’s circadian rhythm, based on a circadian profile established using the estimated values.

10. The system of claim 9, wherein the primary physiological parameter or biomarker is a core body temperature of the user, a cortisol level of the user, and/or a melatonin level of the user.

11. The system of claim 9, wherein the first sensing arrangement comprises an ingestible sensor.

12. The system of claim 9, wherein the first sensing arrangement comprises a patch or textile configured to be worn on a body of the user.

13. The system of claim 9, wherein the second sensing arrangement is provided in at least one wearable device, wherein the at least one wearable device comprises one or more of: a smart watch, a smart ring, an ear wearable device, a band configured to be worn, an eye mask, a collar, and headphones.

14. The system of claim 9, wherein the controller is configured to determine a correlation model based on at least one of the correlations determined using the first data and the second data measured over the initialization period.

15. The system of claim 14, wherein the controller is configured to store the first data and the second data measured over the initialization period in relation to the user.

16. The system of claim 15, wherein the controller is configured to update the correlation model based on the stored first data and the second data, new second data acquired using the second sensing arrangement over a subsequent period, and new first data acquired over the subsequent period in relation to a physiological parameter or levels of a biomarker which has a circadian cycle.

17. The system of claim 16, wherein the new first data is associated with a primary parameter or biomarker with which the first data acquired over the initialization period is associated.

18. The system of claim 9, comprising a mobile computing device configured to receive data from the first sensing arrangement and provide the received data to the controller.

19. The system of claim 18, wherein the mobile computing device comprises a memory storing machine readable program, and a processor configured to execute the machine readable program to:
receive user input to initiate profiling of the user’s circadian rhythm;
cause the mobile computing device to output an instruction to operate the first sensing arrangement to acquire the first data; and
receive the first data from the first sensing arrangement.

20. A non-transitory computer-readable storage medium comprising instructions stored thereon that, when executed by one or more processors of a computing system, cause the computing system to perform operations for profiling a user’s circadian rhythm, the operations comprising:
receiving first data acquired using a first sensing arrangement configured to measure a primary physiological parameter or a biomarker known to be associated with a circadian rhythm;
receiving second data acquired using a second sensing arrangement comprising one or more monitoring sensors, each configured to measure a secondary physiological parameter or biomarker associated with the user;
determining one or more correlations between the first data and the second data, wherein the first data and the second data are acquired over an initialization period;
receiving monitoring sensor data from the second sensing arrangement over a subsequent period after the initialization period;
estimating values of the primary physiological parameter or biomarker based on (i) the determined one or more correlations and (ii) the monitoring sensor data; and
profiling the user’s circadian rhythm based on a circadian profile established using the estimated values.
